(19) United States
(12) Patent Application Publication
(10) Pub. No.: US 2015/0182729 A1
(43) Pub. Date: Jul. 2, 2015

(54) CATHETER SYSTEM FOR DELIVERY OF SOLUTIONS

(71) Applicant: EMORY UNIVERSITY, Atlanta, GA (US)

(72) Inventors: Jakob Vinten-Johansen, Grayson, GA (US); Bruce W. Copeland, Tucker, GA (US)

(21) Appl. No.: 14/409,930

(22) PCT Filed: Mar. 12, 2013
(86) PCT No.: PCT/US2013/030412

§ 371 Date: Dec. 19, 2014

Related U.S. Application Data
(60) Provisional application No. 61/663,744, filed on Jun. 25, 2012.

(51) Int. Cl. A61M 25/02; A61M 25/01; A61M 25/04; A61M 25/00

(52) U.S. Cl. CPC A61M 25/02; A61M 25/0084; A61M 25/01; A61M 25/04; A61M 2025/0286; A61M 2025/0004

(57) ABSTRACT

A catheter system may include an adapter and a catheter configured to improve the field of vision and support of a catheter during surgical and interventional procedures, such as open-heart procedures. The adapter may include at least one suture securing member configured to releasably secure a suture with respect to a catheter. The adapter may include at least one grip section configured to be gripped by digit(s) of a user. The catheter may include a platform member that includes at least one connecting member configured to receive and support a lumen of a catheter.

### CROSS REFERENCE TO RELATED APPLICATION

This application claims priority to U.S. Provisional Application Ser. No. 61/663,744 filed on Jun. 25, 2012, which is hereby incorporated by this reference in its entirety.

### BACKGROUND

For some cardiac surgery procedures, such as coronary artery bypass graft surgery (CABG), it is preferable or necessary to stop the beating of a heart for a period of time so that the surgery can be performed while on cardiopulmonary bypass. The heart may be stopped by administering a solution, such as a cardioplegia solution, to the heart.

Typically, when open heart surgery is performed, the chest is opened using a median sternotomy to gain surgical access to the heart. This also allows access to the aorta for cross clamping, which is important for standard methods of administering cardioplegia because it effectively separates the heart circulation from that of the rest of the body. Before stopping the heart, the patient is prepared by placing an arterial cannula and a venous cannula which are connected to a cardiopulmonary bypass (CPB) system. The CPB system takes over the functions of the heart and the lungs of the patient by oxygenating and pumping the blood to the rest of the body. Once the CPB system is connected and started, the ascending aorta can be cross clamped to isolate the coronary arterial circulation from the rest of the systemic arterial circulation. Then, cardioplegic arrest can be induced by infusing a prescribed quantity of a cardioplegic solution through a catheter that is generally placed thru the wall of the ascending aortic root.

Conventional cardioplegia techniques require the use of a catheter lumen through which solutions can be delivered, suture(s), rubber sleeves (rummels), and associated hemostats for securing the suture(s) and for maintaining hemostasis around the insertion point of the catheter. The sheer number of “devices” typically required to accomplish the administration of the cardioplegia solution can “clutter” the operative field, and thereby can obstruct access and visibility required to perform certain surgical procedures, as well can be cumbersome to move when the heart is repositioned to gain access to posterior or lateral coronary arteries.

Thus, there is a need for a catheter system constructed that does not interfere with the visibility of the surgical field. Such a catheter system organizes and reduces the clutter in the operative field and simplifies movement of the catheter when the heart is repositioned.

### SUMMARY

The disclosure relates to catheter systems, adapters, and catheters. The catheter systems, adapters, and catheters are configured to stabilize a catheter without interfering with the visibility of or access to the surgical field.

In some embodiments, the catheter system may include a catheter having a catheter body, the catheter body having a first end, a second end, a length between the first end and the second end; and an adapter disposed on the catheter body substantially perpendicular to the length. The adapter may include at least one suture guiding member configured to releasably secure and/or maintain a suture in tension. The at least one suture guiding member includes at least one secure mechanism.

In some embodiments, the adapter may include a body including a first surface, an opposing second surface, and a length therebetween. The adapter may include at least one opening (also referred to as a channel) extending between the first and second surfaces and configured to receive at least a portion of a catheter. In some embodiments, the adapter may be fixedly disposed and/or removably disposed along a catheter body.

In some embodiments, the adapter may include at least one grip section disposed on one side of the body. In some embodiments, the adapter may include a first grip section and a second grip section. The body may be between the first grip section and the second grip section.

In some embodiments, each grip section may include a gripping surface configured to be held or gripped by a user's finger(s). The surface(s) may be configured so that at least one finger of a user is enclosed. In some embodiments, the surface of the first grip section may be the same as the surface of the second grip section. In other embodiments, the surface of the first grip section may be different from the surface of the second grip section.

In some embodiments, the adapter may include a first suture securing member and a second suture securing member. Each of the suture securing members may include a first surface, an opposing second surface, and an opening therebetween.

In some embodiments, the secure mechanism may be disposed on at least one of the surfaces within the opening. The opening member may be configured to releasably tension a suture.

In some embodiments, the first surface may be configured to maintain tension of a suture. In some embodiments, the first surface may be configured to maintain the tension of a suture held by a suture securing member.

In some embodiments, the adapter may include a guide member configured to guide a suture to the opening and/or secure mechanism. In some embodiments, the adapter may include a guide member for each suture securing member. In some embodiments, the guide member may be disposed below the respective suture securing member.

In some embodiments, the guide member may include a guide surface. The guide surface may be a tapered surface.

In some embodiments, the adapter may include a least one member configured to reduce the tension exerted by a suture on the suture securing member. The member may be disposed above the suture securing member. In some embodiments, the member may project from an outer surface substantially perpendicular to the first and second surfaces of the adapter body. In some embodiments, the member may have a pin-like shape. In some embodiments, the member may have a hook-like shape.

In some embodiments, the catheter may include at least a first lumen configured to receive a needle and a second lumen configured to receive a stylet. In some embodiments, the catheter may include a third lumen. The first lumen being disposed between the second and third lumens.

In some embodiments, the catheter may include at least one additional lumen. The lumen may be disposed substantially perpendicular to the second and third lumens and adjacent to the first lumen.

In some embodiments, one or more additional lumens may be configured to deliver pressure, to provide an outlet for air, gas, debris and/or a liquid, to provide an inlet for air, gas, debris and/or a liquid, or any combination thereof. In some embodiments, the catheter may include a fourth lumen and a fifth lumen configured to deliver pressure and/or to provide an inlet/outlet for air, gas, debris, or liquid, respectively.

In some embodiments, the catheter may include a platform that is disposed adjacent to an end of the catheter. The platform may be fixedly or removably disposed. The platform may be configured to support the catheter body and/or maintain and/or stabilize a position of the catheter with respect to an insertion site.

In some embodiments, the platform may include a base that includes a first surface and a second opposing surface. In some embodiments, the base may include at least one opening that extends between the first and second surfaces. In some embodiments, the base may include at least one connecting member that protrudes from the first surface. The connecting member may be configured to maintain an alignment or overlying registration of a lumen with an opening. The connecting member may be configured to surround an opening.

In some embodiments, the platform may include three openings. In some embodiments, the platform may include three connecting members. The first, second and third connecting members may be configured to receive the first, second, and third lumens, respectively.

In some embodiments, the platform may include a fourth connecting member. The fourth connecting member may be configured to receive a pressure lumen.

In some embodiments, the platform may further include at least one support member configured to protrude from the second surface. The support member may be disposed opposite of the first connector. The support member may be configured to maintain and/or stabilize a position of an instrument disposed within the first lumen. The instrument may include but is not limited to a needle.

In some embodiments, the support member may have a taper elongated shape and may include an opening disposed at one end.

In some embodiments, the platform may include at least one protruding tab. The protruding tab may include a barb on the protruding end. In some embodiments, the platform may include two protruding tabs, each tab being disposed on the first surface at opposing ends.

In some embodiments, the catheter system may include at least one stylet configured to be movable within a lumen. In some embodiments, the catheter system may include two stylets.

In some embodiments, each stylet may include a first end, an opposing second end, and a length therebetween. In some embodiments, the stylet may include an obstructing member disposed along the length. The obstructing member may be configured to control the proximal movement with respect to a lumen. In some embodiments, the stylet may be configured to control the proximal movement of the stylet towards an insertion site. In some embodiments, the obstructing member may be configured to prevent the stylet from moving further proximally when the obstructing member is disposed against an edge or opening of the lumen. In some embodiments, the stylet may have an open curved shape.

In some embodiments, the disclosure may relate to a catheter system. The catheter system may include a catheter, the catheter including a catheter body having a first end, an opposing second end, and a length therebetween, the catheter including at least one lumen disposed at least partially along the length; an adapter; and a platform. The adapter may be configured to secure a tension of a suture pulled through a lumen, the adapter being disposed on at least a portion of the catheter body, substantially perpendicular to the length, the adapter being disposed substantially adjacent to the first end. The adapter may include a body, the body including a first surface, a second surface opposing the first surface, a length between the first and second surfaces, the body including an opening extending between the first and second surfaces; at least one grip section disposed adjacent to the opening, the grip section include a gripping surface configured to be held by or gripped by a user's fingers; and at least one suture securing member, the at least one suture securing member including a first surface, a second surface opposing the first surface, and an opening, the at least one suture securing member including at least one secure mechanism disposed on one of the surfaces, the secure mechanism being configured to releasably hold tension of a suture. The platform may be configured to stabilize a position of the catheter with respect to an insertion site, the platform being substantially adjacent to the second end. The catheter may include at least two lumens.

In some embodiments, the catheter system may include a needle. The needle may include a puncture member disposed at one end.

In some embodiments, the disclosure may relate to kits. The kits may be sterilized and configured for single use. The kit may include an adapter according to embodiments. In some embodiments, the kit may include a catheter. In some embodiments, the kit may further include stylet(s) and/or a needle. In some embodiments, the kit may further include at least one of sutures or pladgets.

Additional advantages of the disclosure will be set forth in part in the description which follows, and in part will be obvious from the description, or may be learned by practice of the disclosure. The advantages of the disclosure will be realized and attained by means of the elements and combinations particularly pointed out in the appended claims. It is to be understood that both the foregoing general description and the following detailed description are exemplary and explanatory only and are not restrictive of the disclosure, as claimed.

### BRIEF DESCRIPTION OF THE DRAWINGS

The disclosure can be better understood with the reference to the following drawings and description. The components in the figures are not necessarily to scale, emphasis being placed upon illustrating the principles of the disclosure.

FIG. 1 is a view of a catheter system according to embodiments;

FIG. 2 is an enlarged partial view of FIG. 1;

FIG. 3 is an enlarged partial view of FIG. 2;

FIG. 4 is a different enlarged partial view of FIG. 1;

FIG. 5 shows a state of operation of the catheter system of FIG. 1;

FIG. 6 shows another state of operation of the catheter system of FIG. 1;

FIG. 7 shows an enlarged partial view of FIG. 6; and

FIG. 8 shows a different enlarged partial view of FIG. 6.

### DESCRIPTION OF THE EMBODIMENTS

The following description, numerous specific details are set forth such as examples of specific components, devices, methods, etc., in order to provide a thorough understanding of embodiments of the disclosure. It will be apparent, however, to one skilled in the art that these specific details need not be employed to practice embodiments of the disclosure. In other instances, well-known materials or methods have not been described in detail in order to avoid unnecessarily obscuring embodiments of the disclosure. While the disclosure is susceptible to various modifications and alternative forms, specific embodiments thereof are shown by way of example in the drawings and will herein be described in detail. It should be understood, however, that there is no intent to limit the disclosure to the particular forms disclosed, but on the contrary, the disclosure is to cover all modifications, equivalents, and alternatives falling within the spirit and scope of the disclosure.

The terms “distal” and “proximal” used herein with respect to the catheter system, catheter, adapter and features are with respect to the position of the catheter system, catheter, adapter and features when in use. “Proximal” indicates an end of the catheter system, catheter, adapter or a feature thereof closest to, or a direction towards the insertion site, and “distal” indicates an end of the catheter system, catheter, adapter or a feature thereof farthest from, or a direction away from the insertion site. “Insertion site” refers to any site or region of a subject, human or animal, into which the catheter is intended to be inserted, such as a tissue of an organ or muscle. For example, the insertion site may include but is not limited to an ascending aorta of a heart.

The catheter system, catheter, and adapter according to embodiments address potentially problematic obstruction of the operative field that can be associated with conventional cardioplegia techniques. It would be understood that the catheter system, catheter, and/or adapter, as well as components thereof, according to embodiments are not limited to open-heart surgeries and may be used and intended for other purposes. The catheter system, catheter, and/or adapter may be configured to measure aortic pressures during any procedure. The adapter and/or stylet system may be used to retrieve and tighten any suture holding a catheter in any vessel, e.g., a sheath for vascular access during cath-lab intervention and/or percutaneous procedures, such as catheter-based diagnostics, angioplasty and stent placement. The catheter system, catheter, and/or adapter may be used in a procedure in which vascular access catheters or other devices are secured by sutures, including but not limited to venous return catheters in cardiopulmonary bypass, LVAD implantations, as well as left ventricular apical access devices.

In some embodiments, the catheter system and catheter may have a longer overall body length, as compared to conventional catheters. This can reduce manipulation of the aortic tack site sutures in the well of the incision in minimally invasive or robotic surgery.

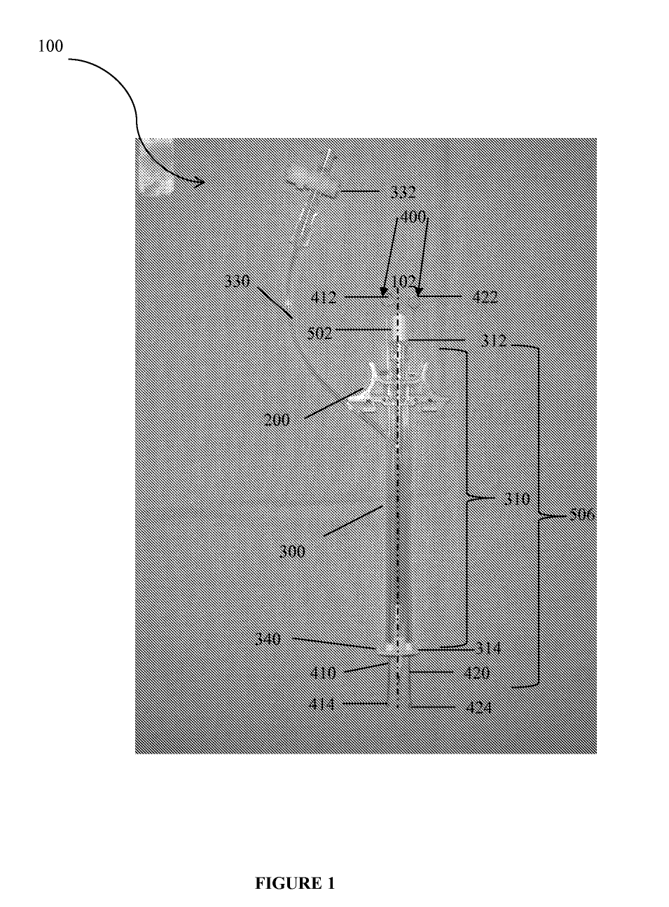

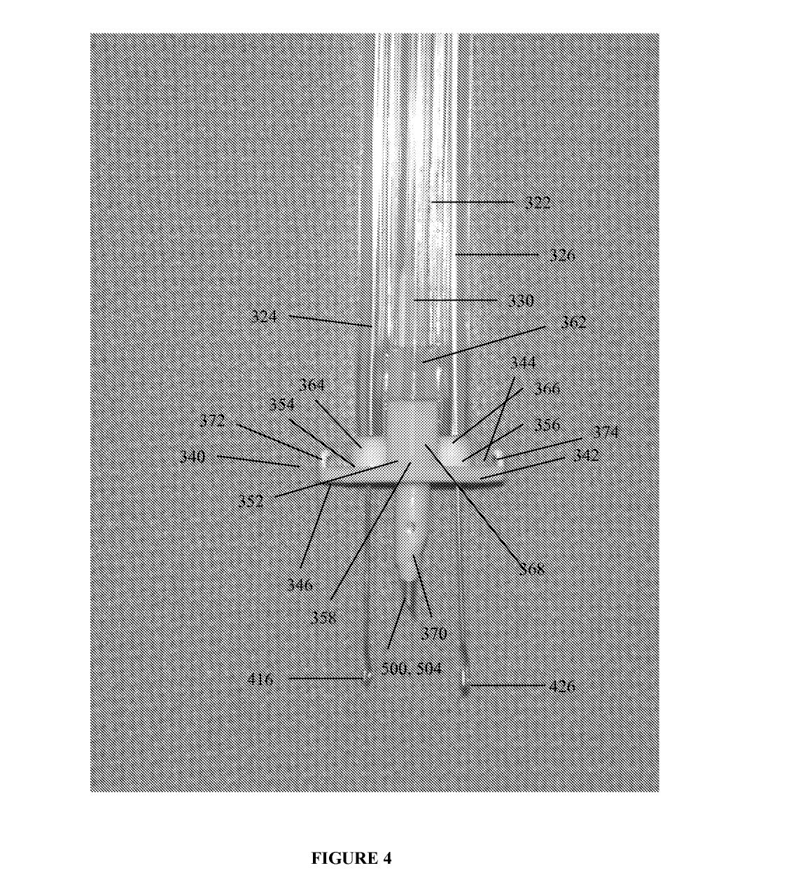

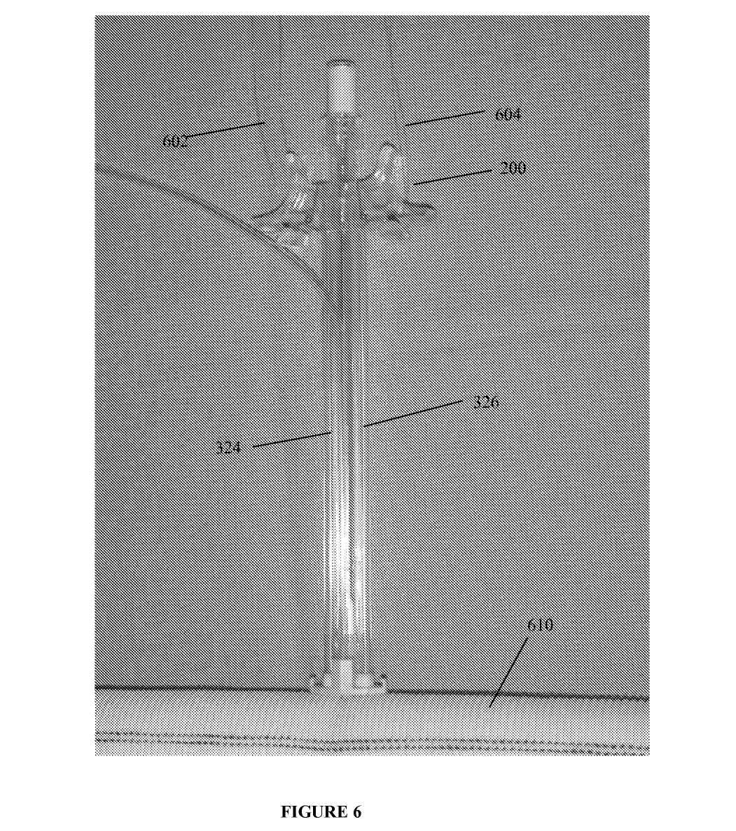

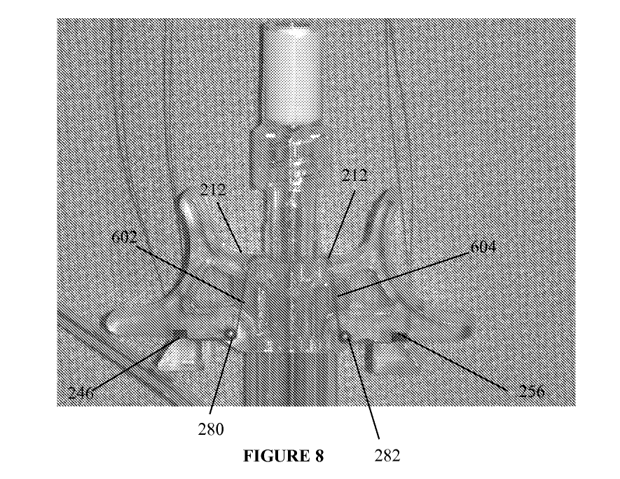

FIGS. 1 through 8 show a catheter system 100 according to embodiments. As shown in the figures, the catheter system 100 may include an adapter 200. In some embodiments, the catheter system 100 may further include a catheter 300. In some embodiments, the catheter system 100 may further include at least one stylet 400. In some embodiments, the catheter system 100 may include a needle 500.

It will be understood that the adapter 200, the catheter 300, the stylet 400 and the needle 500 may be provided separately and/or in any combination. Also, the catheter system 100 may include a different adapter, catheter, stylet and/or needle.

The catheter systems, adapters, and catheters may be configured for invasive surgery procedures, as well as less invasive surgery procedures (for example, procedures that use mini-sternotomy incisions or thoracotomy incisions). The configuration, shape, and length of the catheter and/or adapter may be modified to accommodate different surgery procedures. For example, the catheter body may be lengthened to accommodate less invasive surgery procedures that use mini-sternotomy incisions or thoracotomy incisions.

### Catheter

In some embodiments, the catheter 300 may include at least one lumen. In some embodiments, the catheter 300 may include a catheter body 310. The catheter body 310 may include a first end 312 and a second end 314 (closer to the insertion site). In some embodiments, the catheter body 310 may include more than one lumen. The lumen(s) may extend entirely and/or partially between the first end 312 and the second end 314.

In some embodiments, the catheter body 310 may include at least two lumens. In other embodiments, the catheter 300 may include at least three lumens. In further embodiments, the catheter 300 may include more than three lumens. The catheter 300 may include any number of lumens. At least one of the lumens may be configured to allow movement of a needle and/or flow of a fluid therethrough and at least another one of the lumens being configured for movement of a stylet therethrough. The lumen(s) may be parallel with a central axis 102.

In some embodiments, the lumens may be partially rigid. In other embodiments, the lumens may be at least substantially rigid. In some embodiments, the lumens may be integrally formed, separately formed, or a combination thereof.

In some embodiments, the catheter body 310 may include at least one (first) lumen 322 configured to allow movement of a needle and/or flow of a fluid therethrough. The fluid may include but is not limited to a cardioplegia solution, a crystalloid solution, blood, or a combination thereof. In some embodiments, the first lumen 322 may have a length that extends from the first end 312 to the second end 314.

In some embodiments, the catheter body 310 may include at least one (second) lumen 324 adjacent to the first lumen 322 and configured to allow movement of a stylet. In some embodiments, the catheter 300 may include a third lumen 326 disposed on the opposite side of the first lumen 322 and configured to allow movement of a stylet. In some embodiments, the first lumen 322 may be disposed between the second lumen 324 and the third lumen 326.

In some embodiments, the second and third lumens 324, 326 may extend entirely or partially from about the first end 312 to the second end 314. In some embodiments, one or both of the second and third lumens 324, 326 may originate from about the second end 314, extend towards the first end 312 and terminate between the first end 312 and the second end 314.

In some embodiments, the catheter body 310 may include at least one additional (also referred to as a “fourth”) lumen 330. In other embodiments, the catheter body 310 may include additional lumens. For example, the catheter body 310 may include a pressure lumen and/or a venting lumen.

In some embodiments, the lumen 330 may be configured to at least monitor pressure (also referred to as “pressure lumen”), may be configured to provide an inlet/outlet (also can be referred to as “venting lumen”) for an inlet/outlet for air, gas, debris, or liquid, may be configured to deliver drugs, or any combination thereof. The lumen 330 may be disposed adjacent to any one of the lumens. In some embodiments, the lumen 330 may be disposed adjacent to the first lumen 322 and/or one or both of the second and third lumens 324, 326. The lumen 330 may be substantially transverse to at least one of the second and third lumens 324, 326. In some embodiments, the lumen 330 may be disposed substantially adjacent to the first lumen 322 and spaced about 90 degrees from the second lumen 324 and/or third lumen 326.

In some embodiments, the lumen 330 may extend at least partially from the first end to the second end of the first lumen 322. In some embodiments, the lumen 330 may originate from about the second end 314 and extend towards the first end 312. In some embodiments, the lumen 330 may be parallel to the (length of) first lumen 322 along a portion of its length. In some embodiments, the lumen 330 may be configured to extend away from the catheter body 310 between the first end 312 and the second end 314.

In some embodiments, at least one section of the lumen 330 may be configured to be partially or substantially flexible. At least one section of the pressure lumen 330 may be configured to be partially or substantially rigid.

In some embodiments, the lumen 330 may include an elongated pressure line. In some embodiments, the elongated pressure line may be integrated with the lumen 330.

In some embodiments, the lumen 330 may include a connector 332. The connector 332 may be disposed at the (distal) end. The connector 332 may include but is not limited to any conventional luer-type connector. The connector 332 may be configured to receive, in sealed fluid-tight communication, a fluid to be passed through the pressure lumen. This may be configured to remove or prevent air.

In some embodiments, the lumen 330 may be configured to be operatively connected to another device. The device(s) may include but is not limited to: a conventional pressure transducer and monitor that may include but is not limited to a fluid-filled pressure transducer configured to measure the delivery pressure of a fluid, e.g., a cardioplegia solution; a conventional fluid delivery device that may include but is not limited to a delivery device configured to control the delivery of a fluid (e.g., cardioplegia solution and/or drugs adjunct to the cardioplegia solution) based on the received pressure feedback; a conventional pressure monitoring device configured to provide visual and recordable representation of the measured pressure; a suction device configured to remove air and/or debris. For example, if the lumen is attached to a drug delivery device, the position of the lumen allows mixture of a drug at the tip of the catheter (which can be advantageous for drugs that are rapidly degraded by blood or other means, e.g., adenosine and nitric oxide.)

In some embodiments, the lumens may be integrally formed (e.g., within a mold), may be separately formed, or a combination thereof. If any of the lumens are separately formed, they may be fixedly disposed with respect to each other by an adhesive, for example, by glue. The lumens may also be co-extruded.

In some embodiments, the lumens, including the pressure lumen, may be made of any biocompatible material. In some embodiments, the material may include but is not limited to metals, metal alloys, shape memory materials, polymer materials (e.g., polyeutherane or polycarbonate), plastics, thermoplastic elastomers (TPE), synthetic materials, or any combination thereof.

In some embodiments, the catheter 300 may include at least one sensor. The sensor may include but is not limited to a pressure sensor and/or temperature sensor. The pressure sensor may be a solid state sensor.

In some embodiments, the catheter 300 may include a platform member 340. In some embodiments, the platform member 340 may be configured to support the catheter body 300 and/or maintain and/or stabilize the position of the catheter 300, for example, in an upright position, when disposed adjacent to an insertion site. The platform member 340 may be disposed on a catheter substantially adjacent to and/or at the second end 314 of the catheter 300.

In some embodiments, the platform member 340 may include a base member 342. The base member 342 may have an elongated shape. In some embodiments, the base member 342 may extend in a substantially perpendicular direction from an exterior surface of the catheter body 310.

In some embodiments, the base member 342 may have a curved elongated shape. In other embodiments, the base member 342 may have an angular elongated shape. In further embodiments, the base member 342 may have a different shape.

The base member 342 may include a first surface (top) 344 and an opposing second (bottom) surface 346. The second surface 346 may be configured to be disposed on top of and/or face an insertion site, for example, against a portion of the exterior surface of the arterial vessel. In some embodiments, the first surface 344 and the second surface 346 may be substantially parallel to each other.

In some embodiments, the base member 342 may substantially planar. In other embodiments, the base member 342, for example, the second surface 346, may have a different shape complementary to the intended insertion site.

In some embodiments, the platform member 340 may include at least one opening. The opening may extend therethrough the base member 342 from the first surface 344 to the second surface 346. The platform member 340 may include any number of openings, for example, the platform member may include at least two openings, three openings, and more than three openings. In some embodiments, the number of openings may depend on the number of lumens.

In some embodiments, each lumen may be disposed above each respective opening. Each lumen may be in open communication with each opening.

In some embodiments, the platform member 340 may include a first opening 352, a second opening 354, a third opening 356, and a fourth opening 358, as shown in the figures. The first lumen 322 may be disposed above and aligned with the first opening 352, the second lumen 324 may be disposed above and aligned with the second opening 354, the third lumen 326 may be disposed above and aligned with the third opening 356, and the fourth lumen 328 may be disposed above and aligned with the fourth opening 358.

In some embodiments, the platform member 340 may include at least one connecting member configured to receive and support a lumen. The connecting member may be disposed on and protrude from the first surface 344 of the base member 342. In some embodiments, the connecting member may be configured to surround a lumen. In some embodiments, the connecting member may be disposed to border an opening. The connecting member may be configured to maintain the alignment of a lumen with the respective opening and/or support a position of a lumen.

The platform member 340 may include any number of connecting members, for example, the platform member may include at least two connecting members, three connecting members, four connecting members, and more than four connecting members. In some embodiments, the number of connecting members may depend on the number of lumens and/or openings.

In some embodiments, the connecting member may have a circular shape with a hollow center. In other embodiments, the connecting member may have a different shape. In some embodiments, the shape of the connecting member may depend on the shape of the lumen and/or opening in the platform member.

In some embodiments, the platform member 340 may include a first connecting member 362, a second connecting member 364, a third connecting member 366, and a fourth connecting member 368, as shown in the figures. The first lumen 322, the second lumen 324, the third lumen 326, and the fourth (pressure) lumen 328 may be disposed within the first connecting member 362, the second connecting member 364, the third connecting member 366, and the fourth connecting member 368, respectively, so as to maintain the position of each lumen in an overlying registration (e.g., the alignment) with the respective openings 352, 354, 356, and 358.

In some embodiments, the platform member 340 may include at least one support member disposed on and protrude from the second surface 344 of the base member 342. The support member may be configured to maintain the position of and stabilize an instrument, for example, a needle. In some embodiments, the support member may oppose the respective connecting member and protrude in the opposite direction.

The support member may have an elongated tapered shape with an opening. The size of the opening may vary based on the instrument. The length of the support member may vary based on the insertion site. In other embodiments, the support member may have a different shape. The shape of the support member may vary based on the instrument.

In some embodiments, the platform member 340 may include a support member 370 disposed at the first opening 352 opposite of the first connecting member 362, as shown in the figures. In other embodiments, the platform member 340 may include additional support members.

In some embodiments, the support member 370 may be configured to surround the opposite surface of the first opening 352. As shown in the figures, the support member 370 may be configured to support a needle 500.

In some embodiments, the support member 370 may be disposed additionally at the fourth opening 358. The support member 370 may be configured to also surround the opposite surface of the fourth opening 358. The support member 370 may include a separate channel having its own inlet and outlet that communicates with the fourth opening 358 and the opening of the support member 370.

In some embodiments, the platform member 340 may include at least one protruding tab configured to hold the platform member and catheter to a surface of the insertion site, e.g., an aorta or other vessel, by trapping a suture.

In some embodiments, the platform member 340 may include two protruding tabs 372 and 374. In other embodiments, the platform member 340 may include more or less protruding tabs. In some embodiments, the platform member 340 may include one protruding tab. In other embodiments, the platform member 340 may include more than two protruding tabs.

The protruding tabs 372 and 374 may protrude from the first surface 342 of the platform 340 towards the first end 312 of the first lumen 322. In some embodiments, the protruding tabs 372 and 374 may be disposed at opposite ends of the platform 340. In some embodiments, the protruding tab(s) may be disposed at other positions.

In some embodiments, the protruding tabs 372 and 374 may have an elongated shape. In some embodiments, each protruding tab may include a barb (376 and 378, respectively) on the protruding end. In other embodiments, each or all of the protruding tabs 372 and 374 may have a different shape or different configuration that can prevent loss of the suture.

In some embodiments, the platform member (for example, the base, the protruding tabs, the support member, and the connecting members), may be integrally formed (e.g., within a mold), may be separately formed, or a combination thereof. If the features of the platform member are separately formed, they may be combined by an adhesive, for example, by glue. In some embodiments, the platform member and the lumens may be integrally formed or separately formed. In some embodiments, the platform member may be made of any biocompatible material. In some embodiments, the platform member may be made of a flexible material, a rigid material, or a combination thereof. In some embodiments, the material may include but is not limited to metals, metal alloys, shape memory materials, polymer materials (e.g., polyeutherane or polycarbonate), plastics, thermoplastic elastomers (TPE), synthetic materials, or any combination thereof.

### Stylet

In some embodiments, the catheter system 100 may include at least one stylet 400. At least a portion of the stylet being configured to be disposed within and move with respect to one of the lumens, for example, the lumens 324 and 326.

The catheter system 100 may include any number of stylets. In some embodiments, the catheter system 100 may include two stylets, a first stylet 410 and a second stylet 420, respectively. In other embodiments, the catheter system 100 may include more than two stylets. In some embodiments, the number of stylets may vary and depend on the number of lumens configured to receive a stylet.

In some embodiments, the stylet may include a first end, a second end, a length therebetween. As shown in the figures, the first stylet 410 may include a first end 412 and a second end 414; and the second stylet 420 may include a first end 422 and a second end 424.

The stylet may be made of a biocompatible material. The material may include but is not limited to a medical grade metal (e.g., a stainless steel (for example, SS303 and SS304 stainless steel), or other suitable material, such as, for example, a polymer).

In some embodiments, the stylet may include a suture capture member at the second end. The suture capture member may have an open-curved shape, like a hook. In other embodiments, the suture capture member may have a different shape. The suture capture member may be configured to grasp a suture disposed within the insertion site, e.g., aortic arch. As shown in the figures, the first stylet 410 may include a suture capture member 416 disposed at the second end 414 and the second stylet 420 may include a suture capture member 426 disposed at the second end 424.

In some embodiments, the stylet may include a handling member disposed at the opposing end, the first end, configured to facilitate the ease of handling the stylet. In some embodiments, the handling member may be a loop. In other embodiments, the member may have a different shape. As shown in the figures, the first stylet 410 may include a handling member 418 disposed at the first end 412 and the second stylet 420 may include a handling member 428 disposed at the first end 422.

In some embodiments, the stylet may include an obstructing member disposed between the first end and the second end. The obstructing member may be disposed above the lumen when the stylet is provided within the lumen. The obstructing member may be configured to control the movement of the stylet with respect to the lumen. The obstructing member may be configured to restrict the movement of the stylet with respect to the lumen. In some embodiments, the obstructing member may be configured to control proximal movement of the stylet towards an insertion site. The obstructing member may be configured to restrict proximal movement by preventing the stylet from moving further proximally when the obstructing member is disposed against an edge of the lumen. In some embodiments, the obstructing member may be configured to control axial movement of the stylet with respect to the lumen. The obstructing member may be configured to restrict the rotation of the stylet with respect to the lumen. The controlled movement of the stylet can facilitate the withdrawal of the stylet from the lumen.

In some embodiments, the obstructing member may be physical and/or structural. In some embodiments, the obstructing member may be a structural feature disposed on the lumen. As shown in the figures, the first stylet 410 may include obstructing member 432 and the second stylet 420 may include second obstructing member 434. In some embodiments, the obstructing member 432, 434 may have an open curved, protruding shape that protrudes substantially transverse to the opening of the lumen (or substantially perpendicular to the central axis 102). In other embodiments, the obstructing member may have a different shape.

In some embodiments, the obstructing member may be made of material configured to control the movement of the stylet with respect to the lumen. For example, the obstructing member may be made of a magnetic material that restricts the movement of the stylet due to the magnetic force when disposed above a corresponding magnetic material on the lumen.

### Needle

In some embodiments, the needle 500 may be disposed in and move with respect to the first lumen 322. The needle 500 may be any conventional needle. The needle 500 may be a solid or a hollow needle. The needle may include an opening disposed within the shaft of the first lumen 322. In some embodiments, the needle 500 may include an end 502 with a lock, an opposing end 504 with a puncture member, and a shaft 506 therebetween.

### Adapter

In some embodiments, the adapter 200 may be disposed along a length of a catheter or catheter body. In some embodiments, the adapter 200 may be removably disposed along a length of a catheter or a catheter body. In other embodiments, the adapter 200 may be fixedly disposed along a length of a catheter or catheter body.

In some embodiments, the adapter 200 may be configured to be disposed closer towards the proximal end (further from the insertion site) of the catheter (e.g., end 312 of the catheter 300) than the distal end. The adapter 200 may be configured to assist in the placement of a catheter and/or to releasably secure and fix a portion of a suture relative to a catheter.

In some embodiments, the adapter 200 may include an adapter body (also referred to as “body”) 210. The body 210 may include two opposing surfaces 212 and 214. In other embodiments, the surfaces 212 and 214 may have the same shape and/or length. In other embodiments, the surfaces 212 and 214 may have different shapes and/or lengths. In some embodiments, the surfaces 212 and 214 may be parallel to each other, as shown in the figures. In other embodiments, the surfaces 212 and 214 may be not parallel to each other, for example, the surfaces 212 and 214 may be substantially oblique or perpendicular with respect to each other.

In some embodiments, the body 210 may have a length that extends between and/or may be substantially perpendicular to the first surface 212 and the second surface 214. The length may be parallel to the central axis 102. The first surface 212 and the second surface 214 may have a length that is substantially perpendicular to the central axis 102. The first surface 212 may be configured to maintain tension of a suture.

In some embodiments, one or both of the surfaces 212, 214 may be smooth. In other embodiments, one or both of the surfaces 212, 214 may have a textured surface, for example, a grooved or patterned surface.

In some embodiments, the body 210 may include at least one opening 216 (also referred to as a “channel”) that extends along the length of the central body 210 between the first surface 212 and the second surface 214. The opening 216 may be configured to receive all or a portion of a catheter body. The catheter body may include but is not limited to the catheter body 300 described in the embodiments and shown in the figures. The size and shape of the opening 216 may vary and may depend on the size and shape of the catheter body. For example, the opening 216 may be configured to the shape of each lumen of the catheter body.

In some embodiments, the adapter 200 may include at least one section having an ergonomic shape. In some embodiments, the at least one section (also referred to as a “grip section”) may be configured to be gripped by digit(s) of a user. In some embodiments, the adapter 200 may include more than one grip section. In some embodiments, the grip section(s) may be disposed adjacent to the body 210.

As shown in FIGS. 1 through 7, the adapter 200 may include two grip sections, first and second grip sections 220 and 230, respectively. The grip sections 220 and 230 may be disposed on the sides of the central body 210. In some embodiments, the adapter 200 may include more than two sections.

In some embodiments, the first and second grip sections 220 and 230 may be disposed substantially parallel to the length of the central body 210. In some embodiments, the first and second grip sections 220 and 230 may be symmetric. In other embodiments, the first and second grip sections 220 and 230 may be asymmetric. In some embodiments, the first and second grip sections 220 and 230 may have the same shape. In other embodiments, the first and second grip sections 220 and 230 may have a different shape.

In some embodiments, the adapter 200 may include at least one (gripping) surface configured to be held or gripped by a user's finger(s). In some embodiments, the surface may be configured so that at least one finger of a user is enclosed.

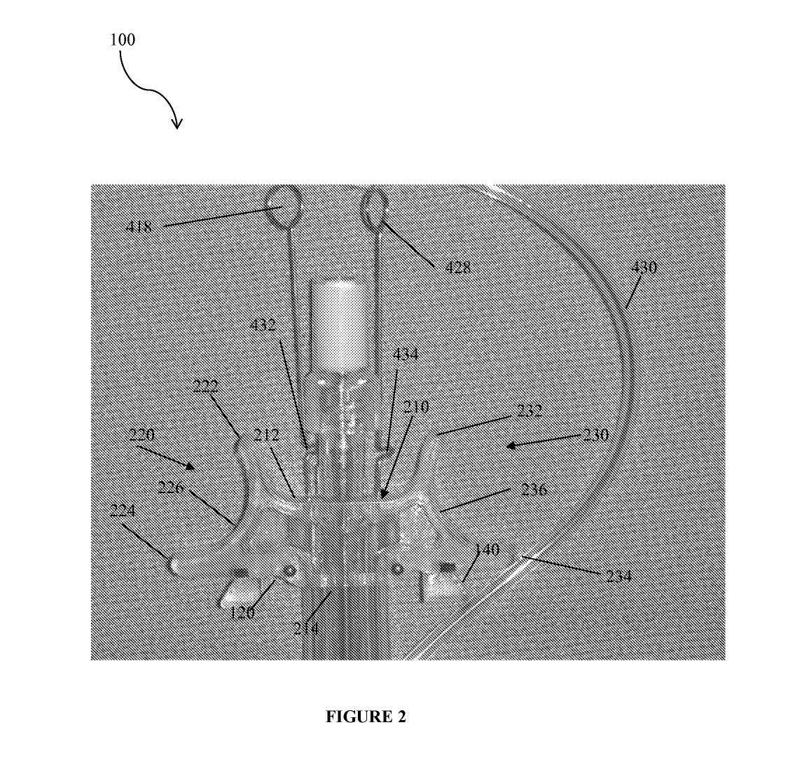

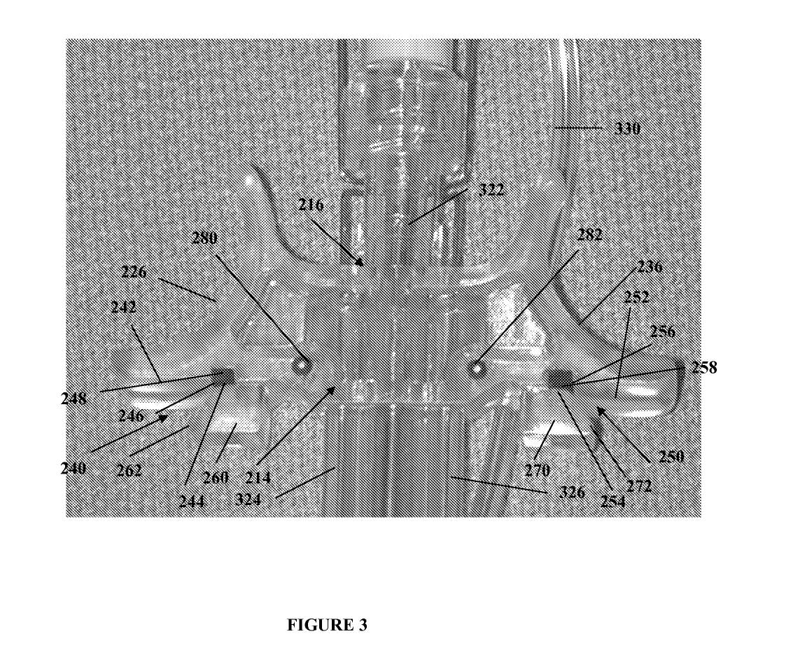

In some embodiments, the adapter 200 may include a surface disposed at least one grip section. In some embodiments, the adapter 200 may include a surface disposed at each grip section. As shown in FIGS. 2 and 3, the adapter 200 may include at least first surface 226 disposed at the first grip section 220 and a second surface 236 disposed at the second grip section 230.

In other embodiments, one, some, or all section(s) may include more than one (gripping) surface disposed at least one grip sections. The number of surfaces and shapes may vary and be configured to depend on the intended grip of the user. For example, if the adapter is configured to be gripped by two fingers, the adapter may include two adjacent surfaces disposed at one section.

In some embodiments, the shape and length (or circumference) of the surface may vary and may depend on the intended grip of the user. In some embodiments, the surface may be curved. In some embodiments, the surface may be concaved. In some embodiments, the surface may be configured to form a pocket at the section. In some embodiments, the surface may be curved or substantially curved so as to form an arc that exceeds ninety degrees. In other embodiments, the surface may have a different shape.

As shown in the figures, each of the sections 220 and 230 may include first ends 222 and 232 and second ends 224 and 234, respectively. In some embodiments, the second ends 224 and 234 may extend further than the first ends 222 and 232, respectively, in a direction substantially perpendicular to the central axis 102. The first ends 222 and 232 may extend beyond the first surface in a direction parallel to the length of the body 210.

In some embodiments, the adapter 200 may include at least one suture securing member (also referred to as “suture secure member”) configured to releasably secure a suture. The at least one suture securing member may be configured to releasably maintain a suture in tension. The suture securing member may be configured to hold a catheter in a vessel, for example, a sheath for vascular access during cath-lab interventions, such as catheter-based diagnostics, angioplasty and stent placement. The suture securing member may be configured to hold the platform of the adapter against the surface of the insertion site, for example, an exterior surface of a vessel, by securing the suture(s).

In some embodiments, the adapter 200 may include two suture securing members 240 and 250. The adapter may include any number of suture securing members. In other embodiments, the adapter may include more or less than two suture securing members. The number of suture securing members may depend on the number of lumens configured for sutures of a catheter.

The suture securing members 240 and 250 may be disposed on adjacent to the first and second grip sections 220 and 230. The suture securing members 240 and 250 may be disposed below the first and second grip sections 220 and 230, respectively. In other embodiments, the suture securing members 240 and 250 may be disposed at other positions on the body 210.

In some embodiments, each suture securing member may include two opposing surfaces and an opening formed therebetween. As shown in the Figures, each suture securing member 240 and 250 may include a first surface 242, 252, respectively; a second surface 244, 254; and an opening 246, 256 therebetween.

In some embodiments, each suture securing member may include a secure mechanism configured to secure a suture. In some embodiments, the secure mechanism may be configured to temporarily lock the suture in a tension state. The secure mechanism may be disposed on at least one of the surfaces of the suture securing member. As shown in the figures, each suture securing member 240 and 250 may include secure mechanism 248 and 258, respectively.

The secure mechanism may be made of a material that includes but is not limited to an elastomeric material, such as rubber, vinyl, or a soft plastic, as well as a textured material (e.g., a material configured to grip the suture). The material may be an elastically tensioned material.

In some embodiments, the secure mechanism may be a friction-based mechanism. In other embodiments, the secure mechanism may be a non-friction based mechanism, for example, a ratchet-type mechanism, Chinese finger-trap type mechanisms, barbed type mechanisms, as well as other mechanisms. For example, a ratchet-type mechanism may include two opposing surfaces with gripping or textured surface and the two opposing surfaces with gripping or textured surface be configured to engage and secure the suture when the suture is moved (as if it is slipping).

In some embodiments, the suture securing members 240 and 250 may be configured to secure a suture when the suture is disposed across the suture securing member in the opening and in contact with the secure mechanism. The suture securing members 240 and 250 may be configured to release the suture when the suture is removed from the opening 246 and 256, respectively. In some embodiments, the first surface 242, 252 may be a contoured surface configured to guide the sutures into the secure mechanisms 248, 258, respectively.

In some embodiments, the adapter 200 may include at least one suture guiding member disposed adjacent to a suture securing member. The suture guiding member may be configured to guide a suture to the opening of the suture securing member.

In some embodiments, the adapter may include two suture guiding members 260 and 270. The adapter may include any number of suture guiding members. In other embodiments, the adapter may include more or less than two suture guiding members. The number of suture guiding members may depend on the number of lumens suture securing members.

In some embodiments, the suture guiding members 260 and 270 may be disposed below the suture securing members 240 and 250, respectively. In some embodiments, the suture guiding members 260 and 270 may have a protruding shape that protrudes from the bottom (second) surface of the adapter towards the insertion site. In other embodiments, the suture guiding members may have a different shape.

In some embodiments, each of the suture guiding members may have a guiding surface. The guiding surface may be configured to guide a suture to the opening of the respective suture guiding member.

As shown in the figures, each of the suture guiding members 260 and 270 may each include a guiding surface 262 and 272, respectively. The guiding surface 262 and 272 may have a tapered shape. In other embodiments, the guiding surfaces 262 and 272 may have a different shape.

In some embodiments, the guiding surfaces 262 and 272 may be smooth. In other embodiments, the guiding surfaces 262 and 272 may be textured.

In some embodiments, the first surfaces 242 and 252 of the suture securing members 240 and 250, respectively, may be disposed on and/or correspond to the bottom or second surface of the respective grip sections 220 and 230 and/or the body 210. In some embodiments, the first surfaces 242 and 252 may be a contoured surface configured to guide the sutures into the secure mechanisms 248 and 258, respectively. In some embodiments, the second surfaces 244 and 254 may disposed on and/or correspond to the respective suture guiding member 260 and 270.

In some embodiments, the adapter 200 may optionally include at least one member configured to reduce the tension exerted by a suture on the suture securing member. The member may include but is not limited to a projecting member with a round head, such as pin or a hook. The member may be made of any material, for example, a metal and/or plastic material. In other embodiments, the member may have a different shape.

The member may be disposed adjacent and closer to central axis than the suture securing member. In some embodiments, the member may be substantially aligned with the opening.

In some embodiments, the adapter 200 may include two members 280 and 282 configured to reduce the tension exerted by a suture on the suture securing members 240 and 250, respectively. The adapter may include any number of members. The adapter may include more or less members. The number of members may correspond to the number of suture securing members.

In some embodiments, the adapter 200 may not include the members 280 and 282. In these embodiments, the secure mechanism 248, 258 may be disposed on the adapter at a position that substantially corresponds to the position of the members 280 and 282, respectively.

In some embodiments, the adapter, for example, the body, the grip sections, the suture securing members, and the second suture guiding member, may be integrally formed, may be separately formed, or a combination thereof. In some embodiments, the platform member and the lumens may be integrally formed or separately formed.

In some embodiments, the adapter may be made of any biocompatible material. In some embodiments, the adapter may be made of a flexible material, a rigid material, or a combination thereof. In some embodiments, the material may include but is not limited to metals, metal alloys, shape memory materials, polymer materials (e.g., polyeutherane or polycarbonate), plastics, thermoplastic elastomers (TPE), synthetic materials, or any combination thereof.

In some embodiments, some or all parts of the adapter body may be formed with a catheter integrally, separately, or a combination thereof.

### Operation

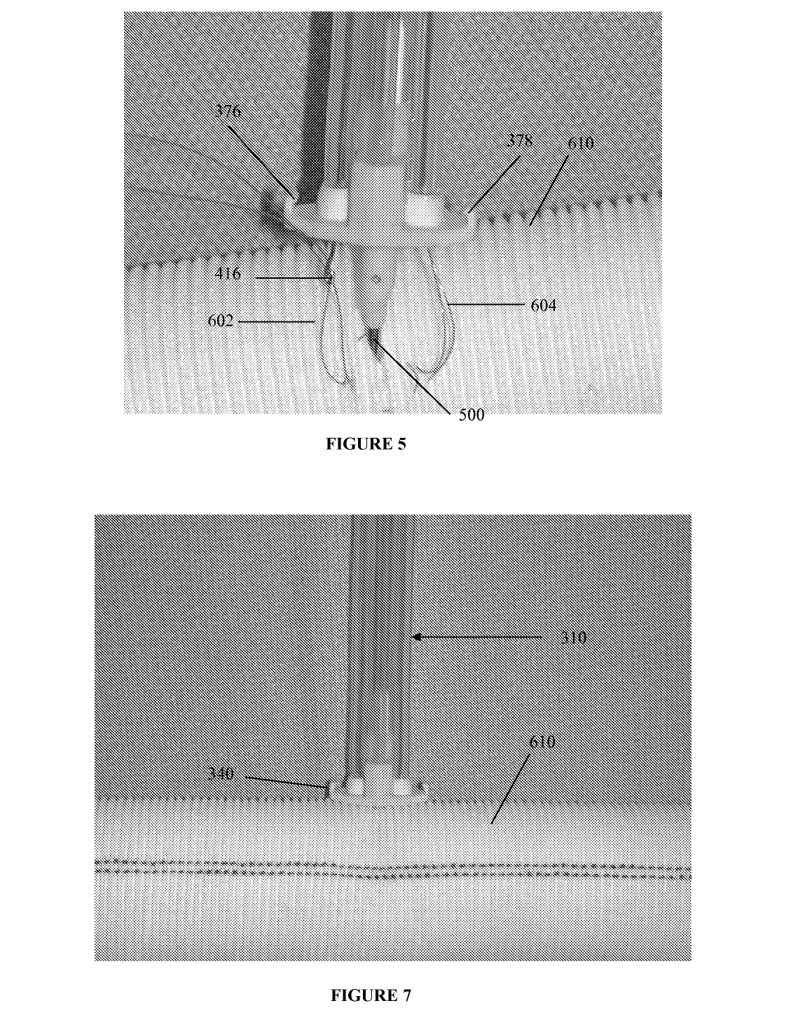

FIGS. 5-8 illustrate an example of operation of a catheter system 100 according to some embodiments. A surgeon may position the catheter using the gripping sections 220 and 230 of the adapter 200 with respect to an insertion site. After the catheter is positioned, the surgeon may first tack purse string stay sutures 602 and 604 to an insertion site 610, as shown in FIG. 5. It will be understood that that a surgeon may also optionally tack one of the purse stay sutures 602 and 604. Then, the stylets 410 and 420 mounted within the respective lumen 320 and 330 may catch the sutures with the respective suture capture members 416 and 426 and guide the sutures 602 and 604 through the respective lumens using the stylets 410 and 420 such that the stay sutures 602 and 604 are drawn up, through and out of the first end of the lumen.

The catheter 300 may then be pushed down so that the puncture end 504 of the needle 500 may be advanced into the insertion site and that the platform member 340 rests on a surface of the insertion site 610, for example, the exterior surface of an arterial vessel.

After the catheter 300 has been properly positioned, the stay sutures 602 and 604 may then be (reversibly) secured relative to the second end of the respective lumens 324 and 326. Each of the stay sutures 602 and 604 may be wrapped around and tensioned against the first surface 212 of the adapter 200. A portion of the sutures 602 and 604 may then be guided into the respective opening 246 and 256 of the suture securing members 240 and 250, respectively, using guide surfaces 262 and 272 respectively. The sutures 602 and 604 may be positioned so as to transverse the opening 246 and 256, respectively. In some embodiments, the sutures 602 and 604 may optionally be tensioned against (wrapped partially or entirely around) members 280 and 282, respectively, before being disposed within the openings 246 and 256. In other embodiments, the sutures 602 and 604 may be disposed within the openings 245 and 256 without being tensioned against members 280 and 282.

By disposing the sutures 602 and 604 within the openings 246 and 256, respectively, the secure mechanisms 246 and 256 exert tension upon the sutures so that they are secured with respect to the catheter 300. The position of the catheter 300 may be stabilized, as well as hemostasis may be maintained. After the position of the catheter 300 is stabilized, procedures may then be performed. For example, the needle 500 may be removed from the central lumen 322 of the catheter body 310 and a conventional cardioplegia supply line may be connected.

When the catheter 300 is to be removed, the sutures 600 may be released from the adapter 200. The tension may be released by removing the sutures 602 and 604 from the openings 246 and 256.

### Kit

According to some embodiments, one, some or all components of the catheter system may be structured for single use or be disposable. In some embodiments, one, some or all components may be sterilized. According to some embodiments, a portion or combination of the single use items may be sold as a kit.

In some embodiments, the kit may include an adapter according to embodiments. The kit may include at least a catheter according to the embodiments or any conventional catheter. In some embodiments, the kit may include at least one stylet according to the embodiments or any conventional stylet. In some embodiment, the kit may include a plurality of stylets. In further embodiments, the kit may further include a needle according to the embodiments or any conventional needle. In some embodiments, the stylet(s), the needle, and/or the adapter may be premounted into or onto the catheter. In some embodiments, the kit may include suture(s), such as conventional single or double sutures. The kit may also include pledget(s).

While the disclosure has been described in detail with reference to exemplary embodiments, those skilled in the art will appreciate that various modifications and substitutions can be made thereto without departing from the spirit and scope of the disclosure as set forth in the appended claims. For example, elements and/or features of different exemplary embodiments may be combined with each other and/or substituted for each other within the scope of this disclosure and appended claims.

1. An adapter configured to be disposed on a catheter, the adapter including:
a body, the body including a first surface, a second surface opposing the first surface, a length between the first and second surfaces, the body including an opening extending between the first and second surfaces, the opening being configured to a receive portion of a catheter body;
at least one grip section disposed adjacent to the opening, the grip section include a gripping surface configured to be held by or gripped by a user's fingers; and
at least one suture securing member, the at least one suture securing member including a first surface, a second surface opposing the first surface, and an opening, the at least one suture securing member including at least one secure mechanism disposed on one of the surfaces, the secure mechanism being configured to releasably hold tension of a suture.

2. The adapter of claim 1, wherein the adapter includes two grip sections, the sections being disposed on opposite sides of the opening, and two suture securing members, each suture securing member being disposed below a grip section.

3. The adapter of claim 1, further comprising:
at least one guide member configured to guide a suture to the opening of the at least one suture securing member, the guide member being disposed below the grip section.

4. The adapter of claim 3, wherein the guide member includes a guide surface, the guide surface having a tapered shape.

5. The adapter of claim 1, further comprising:
two grip sections, the sections being disposed on opposite sides of the opening, and two suture securing members, each suture securing member being disposed below a grip section; and
at least one two guide members, each guide member being configured to guide a suture to the opening of each suture securing member, each guide member being disposed below each grip section.

6. The adapter of claim 1, wherein the first surface is configured to maintain a tension of at least one suture to be disposed within the opening of the at least one suture securing member.

7. The adapter of claim 1, wherein the opening is configured to receive at least one lumen.

8. The adapter of claim 1, wherein the adapter is configured to be fixedly disposed with respect to a catheter and the adapter is configured to be disposed substantially perpendicular to a length of a catheter.

9. A catheter system, comprising:
a catheter, the catheter including a catheter body having a first end, an opposing second end, and a length between and the first end and the second end, the catheter including at least one lumen disposed at least partially along the length; and
an adapter configured to secure a tension of a suture pulled through a lumen, the adapter being disposed on at least a portion of the catheter body, substantially perpendicular to the length,
the adapter including:
a body, the body including a first surface, a second surface opposing the first surface, a length between the first and second surfaces, the body including an opening extending between the first and second surfaces;
at least one grip section disposed adjacent to the opening, the grip section include a gripping surface configured to be held by or gripped by a user's fingers; and
at least one suture securing member, the at least one suture securing member including a first surface, a second surface opposing the first surface, and an opening, the at least one suture securing member including at least one secure mechanism disposed on one of the surfaces, the secure mechanism being configured to releasably hold tension of a suture.

10. The catheter system of claim 9, wherein the adapter includes two grip sections, the sections being disposed on opposite sides of the opening; and two suture securing members, each suture securing member being disposed below a grip section.

11. The catheter system of claim 9, further comprising:
a guide member configured to guide a suture to the opening, the guide member being disposed below the grip section.

12. The catheter system of claim 11, wherein the guide member includes a guide surface, the guide surface having a tapered shape.

13. The catheter system of claim 9, wherein the first surface is configured to maintain a tension of at least one suture to be disposed within the opening of the at least one suture securing member.

14. The catheter system of claim 9, wherein the catheter includes at least two lumens; and at least one stylet configured to be movable within at least one of the lumens.

15. The catheter system of claim 14, wherein the opening of the body of the adapter is configured to receive a portion of the at least two lumens.

16. The catheter system of claim 14, wherein the stylet includes an obstructing member configured to control proximal movement of the stylet with respect to the lumen.

17. The catheter system of claim 9, further comprising:
a platform configured to stabilize a position of the catheter with respect to an insertion site, the platform being disposed adjacent to an end of the catheter, the platform including a first surface and an opposing second surface.

18. The catheter system of claim 17, wherein the platform includes at least one connecting member disposed on the first surface adjacent to an end, the connecting member being configured to receive and support a lumen of a catheter.

19. A catheter system, comprising:
a catheter, the catheter including a catheter body having a first end, an opposing second end, and a length the first end and the second end, the catheter including at least one lumen disposed at least partially along the length;
an adapter configured to secure a tension of a suture pulled through a lumen, the adapter being disposed on at least a portion of the catheter body, substantially perpendicular to the length, the adapter being disposed substantially adjacent to the first end;
the adapter including:
a body, the body including a first surface, a second surface opposing the first surface, a length between the first and second surfaces, the body including an opening extending between the first and second surfaces;
at least one grip section disposed adjacent to the opening, the grip section including a gripping surface configured to be held by or gripped by a user's fingers; and
at least one suture securing member, the at least one suture securing member including a first surface, a second surface opposing the first surface, and an opening, the at least one suture securing member including at least one secure mechanism disposed on one of the surfaces, the secure mechanism being configured to releasably hold tension of a suture; and a platform configured to stabilize a position of the catheter with respect to an insertion site, the platform being substantially adjacent to the second end.

20. The catheter system of claim 19, wherein the catheter includes at least two lumens.
